(19) United States
(12) Patent Application Publication
(10) Pub. No.: US 2014/0276881 A1
(43) Pub. Date: Sep. 18, 2014

(54) Surgical Staple Guide and Delivery Device

(71) Applicant: Charles TAYLOR, San Antonio, TX (US)

(72) Inventor: Charles TAYLOR, San Antonio, TX (US)

(21) Appl. No.: 14/177,127

(22) Filed: Feb. 10, 2014

Related U.S. Application Data
(60) Provisional application No. 61/777,409, filed on Mar. 12, 2013.

(51) Int. Cl. A61B 17/064; A61B 17/17

(52) U.S. Cl. CPC A61B 17/0642; A61B 17/17
USPC 606/96

(57) ABSTRACT

A surgical device having a first end, a second end, and a middle region. The user may preferably hold the device in the middle region. The first end of the device includes drill guides and the second end of the device includes a staple. The second end further includes a slideable sleeve that facilitates the retention and the release of the staple.

### CROSS REFERENCES TO RELATED APPLICATIONS

This application claims the benefit under Title 35 United States Code §119(e) of U.S. Provisional Application 61/777,409 filed Mar. 12, 2013, the full disclosure of which is incorporated herein by reference.

### BACKGROUND OF THE INVENTION

1. Field of the Invention

In the repair of bones fractures or breaks, surgical methods are used to join two or more bones or fragments together to repair the fracture or break. Similarly, tendons or ligaments may need reattachment to a bone or bone fragment. The joining of bones, bone fragments, tendons and ligaments can be carried out using surgical devices such as bone staples or clamps. The purpose of the staple or clamp is to fix or compress the bone, bone fragment, tendon, or ligament together such that the regions are held together to allow the bone, tendon, or ligament to heal through natural processes and repair the bone fracture or break or detachment of the tendon or ligament.

2. Description of the Related Art

The surgical repair of a bone fracture using a staple or clamp typically involves the use of a drill, a drill guide, drivers, extractors, tampers and mallets. A typical process includes the use of a drill guide placed over the bone followed by the drilling of two holes into the bone and the insertion of the bone staple into the drilled holes. A mallet and/or tamper may be used to hammer the staple so it is flush with the bone. Similar methods can be used to reattach a tendon or ligament. The numerous instruments used during surgical repair of bone fractures and tendon/ligament reattachment are often provided together, e.g., in a kit, and are used many times. The instruments undergo repeated cleaning and sterilization, e.g., by gamma irradiation, electron beam, dry heat, heat or steam autoclave, or gas (e.g., ethylene oxide (EtO)), in accordance with hospital procedures for sterilization.

### SUMMARY OF THE INVENTION

Provided herein is a surgical device having a first and second end, the first end of the device having drill guides and the second end of the device having a staple. The device is optionally a single use device.

The details of one or more embodiments are set forth in the accompanying drawings and the description below. Other features, objects, and advantages of the invention will be apparent from the description and drawings, and from the claims.

### BRIEF DESCRIPTION OF THE DRAWINGS

FIG. 1 is a perspective view of an exemplary surgical device provided herein comprising a bar with drill guides at one end and a slideable sleeve at the opposite end for securing a staple.

FIG. 2A is a perspective view of a surgical device with a slideable sleeve in a first position over the second end of the bar.

FIG. 2B is a perspective view of a surgical device with the slideable sleeve in a second position toward the central portion of the bar of the device.

FIG. 3A is a perspective view of the bar of the device lacking the slideable sleeve.

FIG. 3B is a perspective view of the slideable sleeve.

FIG. 4 is a perspective view of an exemplary surgical device provided herein comprising a bar with four drill guides at one end and a slideable sleeve at the opposite end for securing a staple with four legs.

FIG. 5A is a perspective view of an exemplary surgical device provided herein comprising a slideable sleeve, a step-up staple and a bar with drill guides at one end and, at the opposite end, a groove for receiving a staple.

FIG. 5B is a perspective view of an exemplary surgical device provided herein comprising a bar with drill guides at one end and a slideable sleeve at the opposite end for securing a step-up staple.

FIGS. 6A-6C are perspective, top plan, and side views respectively of a second embodiment of the surgical device of the present invention.

FIGS. 7A-7D are perspective, top plan, side, and bottom plan views respectively of a third embodiment of the surgical device of the present invention.

FIGS. 8A-8D are perspective, top plan, side, and bottom plan views respectively of a fourth embodiment of the surgical device of the present invention.

FIGS. 8E & 8F are end views of the fourth embodiment shown in FIGS. 8A-8D.

FIG. 8G is a cross-sectional view taken along section line A-A′ shown in FIG. 8C.

FIGS. 9A-9D are perspective, top plan, side, and bottom plan views respectively of a fifth embodiment of the surgical device of the present invention.

FIG. 9E is an end view of the fifth embodiment shown in FIGS. 9A-9D.

FIG. 9F is a cross-sectional view taken along section line A-A′ shown in FIG. 9C.

FIGS. 10A-10C are detailed views of an alternate sleeve embodiment of the device of the present invention shown with a staple incorporated onto the device.

FIGS. 11A-11C are detailed views of the alternate sleeve embodiment shown in FIGS. 10A-10C but with the staple removed from the device.

### DETAILED DESCRIPTION OF THE DRAWINGS

The present application provides a surgical device that simplifies the need for a large number of instruments used during surgical repair of bones, ligaments, tendons, or joints. In one aspect, the provided surgical device eliminates the need for repeated cleaning and sterilization of the instruments and improves efficiency during surgery by providing a multi-use device. The provided surgical device of the present application combines a drill guide and a staple onto a single device, wherein the drill guides and staple correspond so that the drill guides are appropriately spaced for the staple dimensions and configuration. The device is optionally made of a disposable material and is discarded after a single use.

FIG. 1 shows a view of the device that comprises a bar 4 and a slideable sleeve 5. The bar has a first end 1, a second end 2, and a central portion 3. A staple, which is not shown, is held in place in slot 300 by a slideable sleeve 5 positioned over the second end 2 of the bar 4. In the pictured embodiment, metal drill guides 120 are in position in the drill guide holes 100 at the first end 1 of the bar 4.

FIGS. 2A & 2B show a perspective view of the device with a staple 110 in position. FIG. 2A shows the device with the drill guide holes 100 at a first end 1 of the bar 4 and, at the second end 2 of the bar 4, the slideable sleeve 5 holds the staple 110 in position. As shown, the slideable sleeve is in a first position 200 over the first end 1 of the bar 4. A central portion 3 of the bar 4 extends between the first end 1 and second end 2 of the bar 4. The slideable sleeve 5 is configured to cover the middle portion of the staple 112 and to maintain the legs 111 of the staple in a position approximately parallel to the drill guides when the sleeve is in the first position 200. FIG. 2B shows the device with the slideable sleeve 5 in a second position 201 toward the central portion 3 of the bar 4 so as to allow release of the staple 110.

FIGS. 3A & 3B show the bar 4 and slideable sleeve 5 separately. FIG. 3A shows the bar 4, having a first end 1 with drill guide holes 100 a central connecting portion 3 and a second end 2. A groove 300 is on the surface of the bar 4 and, more specifically, at the second end 2 of the bar 4. The first end 1 of the bar 4 includes two drill guide holes 100. The width of the groove 300 is approximately the same width as the distance between the two drill guide holes 100. The groove 300 is configured to accept the middle portion 112 of a staple and to allow passage of the slideable sleeve 5 over the middle portion 112 of the staple 110. Thus, the legs 111 of the staple 110 fit into the drill guide holes 100 with one leg 111 in each drill guide hole 100. FIG. 3B shows the slideable sleeve 5. The sleeve is configured to accept the legs 111 of a staple 110 in two notches 301 on either side of a central portion 302. Each notch 301 is configured to accommodate a staple leg 111, and the central portion 302 between the two notches is configured to cover the groove 300 in the second end 2 of the bar 4 when the slidable sleeve 5 is positioned in a first position 200 as shown in FIG. 2A.

FIG. 4 shows the bar 4, having a first end 1 with drill guides 120, a central connecting portion 3 and a second end 2. The first end 1 of the bar 4 includes four drill guides 120. The second end 2 of the bar 4 includes a staple 110 with four legs. The second end 2 also includes a slideable sleeve 5.

FIGS. 5A & 5B show an exemplary device provided herein for a step-up staple. FIG. 5A shows the bar 4, slideable sleeve 5 and step-up staple 110 separately. The bar has a first end 1 with drill guides 120 a central connecting portion 3 and a second end 2. A groove 300 is on the surface of the bar 4 and, more specifically, at the second end 2 of the bar 4, which receives the staple 110. FIG. 5B shows the bar 4, slideable sleeve 5, and step-up staple 110 together. The bar 4 has a first end 1 with drill guides 120 a central connecting portion 3 and a second end 2 containing the step-up staple 110 and the slideable sleeve 5.

Thus, provided is a surgical device comprising a bar 4 with a first end 1 and second end 2 connected by a central arm 3, the first end 1 of the bar 4 comprises two drill guide holes 100 and the second end 2 of the bar 4 comprises a slideable sleeve 5 and a staple 110. The staple has two legs 111 (i.e., a first and second leg) and a middle portion 112 between the two legs 111. The distance between the two drill guide holes 100 is approximately the same as the distance between the two legs 111 of the staple 110 so as to allow passage of the staple legs 111 through the holes drilled according to the pair of drill guide holes 100, wherein the legs 111 of the staple 110 are perpendicular to the length of the bar 4, and wherein the slideable sleeve 5 is positionable in a first position 200 at the second end 2 of the bar 4 over the middle portion 112 of the staple 110 and slideable to a second position 201 toward the first end 1 and over the central arm 3 in part or fully over the central arm 3. Optionally, the bar 4 fits moveably into the slideable sleeve 5. Optionally, the slideable sleeve 5 fully surrounds the bar 4. However, optionally the slideable sleeve covers the staple side and one or more sides of the bar 4 but does not fully surround the bar 4. Optionally the slideable sleeve 5 is held onto the bar 4 by the legs 111 of the staple 110 when the slidable sleeve 5 is in a first position 200 at the second end 2 of the bar 4. Optionally, the staple 110 is releasable from the second end 2 when the slideable sleeve 5 is in the second position 201. Staples 110 are loaded onto the device by sliding the slideable sleeve 5 toward the first end 1 and into the second position 201, placing the staple 110 at the second end 2 of the device, and sliding the slidable sleeve 5 toward the second end 2 and over the middle portion 112 of the staple 110 and into the first position 200 thereby securing the staple 110 onto the device. Optionally, the second end 2 of the device includes a groove 300 into which the staple 110, and, optionally, the middle portion 112 of the staple 110, is placed. The depth of the groove 300 is selected based on the length of the legs 111 of the staple 110 and the dimensions of the slideable sleeve 5. For example, a staple 110 with long legs 111, desired for greater depth of insertion into the bone, tendon, or ligament, may be stabilized in a deeper groove 300 than a staple 110 with shorter legs 111. Similarly, a staple 110 with a longer middle portion 112 would require a groove 300 of greater width.

Optionally the groove 300 for securing the staple 110 is linear when the staple 110 has a linear middle portion 112. However, if the selected staple 110 is non-linear, then the groove will have a similar non-linear configuration so as to allow the middle portion 112 of the staple 110 to be inserted into the groove 300 and to further allow the slideable sleeve 5 to secure the staple 110 in position.

The surgical device provided herein can be made of any material suitable for use as a medical device. Suitable materials include, but are not limited to, metal, plastic, ceramic, and composite materials. Suitable plastic materials include, for example, polystyrene, polypropylene, polyethylene, acrylonitrile-butadiene styrene (ABS), polyurethanes, polyamides, thermoplastic elastomers, polysulfones, polyetheretherketone, polycarbonate, and blends thereof. Optionally, the bar 4, slidable sleeve 5 and drill guide holes 100 are made of plastic. Optionally, the drill guide holes 100 surround a stainless steel lining or drill guides 120. The drill guide holes 100 and the stainless steel linings 120 comprise a cylinder with diameter sufficient for passage of a drill bit so as to allow drilling of two holes of the proper dimensions and locations for insertion of the staple 110 through the drilled holes. Optionally the drill guides 120 fit completely within the drill guide holes 100; however, a portion of the drill guides 120 can extend beyond the drill guide holes 100. Optionally the length of the drill guide 120 is approximately the desired depth of the holes to be drilled for insertion of the staple 110 therein.

The dimensions of the surgical device are of any suitable dimension for its use as a drill guide and staple depositor and of such dimension to stabilize the staple. For example, the distance between the center of the drill guide holes 100 and the length of the middle portion 112 of the selected staple 110 should be approximately equal such that the legs 111 of the staple 110 fit into a pair of holes drilled through the pair of drill guide holes 100. Further by way of example, the length of the bar 4, and particularly the central part 3 of the bar 4, will vary based on the location the staple 110 is to be inserted, with longer dimensions required for more remote or limited surgical access to the point of insertion. For example, the length of the surgical device or bar 4 is from, e.g., 3 to 6 inches. Optionally, the central arm 3 connecting the first end 1 and second end 2 has a width of from 1 to 2 inches and a length from 3 to 6 inches or any value between 1 and 6 inclusive, e.g., 1.2, 1.5, 2, 2.5, and the like. Optionally, the slideable sleeve 5 is from 1 to 3 inches in length or any value between 1 to 3 inches inclusive, e.g., 1.5, 2, 2.5, and the like. Similarly, the size of the notches 301 and central portion 302 in the slideable sleeve 5 will vary based on the dimensions of the staple legs 111.

The surgical device may have one or more than one staple. Bone staples include, but are not limited to, mechanical staples, metal staples, staples that are bent by an instrument, or bendable staples, heat sensitive shape memory alloy staples or memory staples and mechanical elastic bone staples or elastic staples. Optionally, the staple is a memory staple or a mechanical staple. The staple is made of any material suitable for use in surgical repair of bones or joints. For example, the staple is made of titanium alloy, nickel-chromium alloy, nickel-titanium alloy, or stainless steel. Optionally, the staple is made of a single piece of a shape memory alloy, such as, e.g., the nickel-titanium alloy called nitinol. Optionally, the staple is heat activated. Optionally, the staple is made of a different material from the rest of the device. For example, the staple can be made of an alloy and the rest of the device can be made of a plastic.

The staple or clamp can be of any size or shape used in the repair of bones or joints. For example, the staple can be in the shape of a V or an S. The arrangement of the drill guide holes 100 will vary based on the shape of the staple, as will the shape of the groove 300, so as to accommodate the specific staple in the device or in the holes drill using the drill guide holes 100. The size of the staple can be of any size used in the surgical repair of bones and joints. Optionally, the size of the staple is from 4×4 mm to 30×30 mm including any size between 4×4 mm and 30×30 mm. Such dimensions refer to the distance of the middle portion 112 of the staple 110 by the length of the legs 111 of the staple 110. For example, the staple 110 can be of a size 6×4 mm, 9×9 mm, 10×10 mm, 12×12 mm, 20×20 mm, 30×25 mm and the like. Optionally, the size of the staple is from 4×4 mm to 10×10 mm. Optionally, the size of the staple is from 12×12 mm to 20×20 mm. Optionally, the size of the staple is from 20×20 mm to 30×30 mm.

As discussed above, the distance between the two drill guides 100 is approximately the same as the distance between the two legs 111 of the staple 110 and approximately the same distance as the width of the groove 300. Thus, the distance between the two drill guides is, for example, from 4 mm to 30 mm or any distance between 4 and 30 mm inclusive, e.g., 5, 6, 7, 8, 9, 10, 11, 12, 13, 14, 15, 16, 17, 18, 19, 20 mm, and the like.

Further provided is a method of implanting a staple 110 into bone, bone fragment, tendon or ligament. The method includes the steps of providing any one of the surgical devices described herein, placing the first end 1 of the bar 4 comprising the drill guides 100 in a desired position over the bone, bone fragments, tendon, or ligament where a staple 110 is to be inserted, drilling through the drill guides 100 two holes into the bone, bone fragments, tendon, or ligament, placing the legs 110 of the staple 110 into the two drilled holes, and releasing the staple 110 by sliding the slideable sleeve 5 to the second position 201. Optionally the holes can be drilled from either the staple side or the opposite side of the bar, as the drill guides pass through the second end 2 of the bar 4. However, the staple 110 is released from the staple side of the bar 4 once the legs of the staple are partially inserted into the drilled holes. Optionally a second staple 110 is placed in the groove 300 of the second end 2 of the bar 4 and the slideable sleeve 5 is moved to the first position 200 so as to secure a second staple 110 in the same device for subsequent stapling, for example, during the same procedure so as to reload the device with a second staple 110. The device can be reloaded before or after a new set of holes is drilled through the drill guides 100 of the device. Reloading can occur multiple times depending are the number of staples needed in the particular procedure. Optionally the device may be configured as a single use disposable device used as a drill guide, inverted and used to position and place a staple, releasing the staple, and tamping the staple with a mallet portion of the device.

Reference is next made to the remaining Drawing Figures for various alternate preferred embodiments of the surgical device of the present invention providing for alternate structures and geometries for the device.

FIG. 6A is a perspective view showing a second embodiment of the device of the present invention that comprises tool 10 made up primarily of bar 12 and slideable sleeve 14. As with the first embodiment, bar 12 has a first end, a second end, and a central portion. A staple 16 is held in place in a slot (see FIG. 6C) by slideable sleeve 14 positioned over the second end of bar 12. Also shown in FIG. 6A are drill guides 18a & 18b that are positioned in drill guide holes 20a & 20b at the first end of bar 12. FIGS. 6B & 6C show various additional details of this second embodiment of the present invention in top plan and side views of the surgical device.

FIG. 7A is a perspective view showing a third embodiment of the device of the present invention that comprises tool 30 made up primarily of bar 32 and slideable sleeve 34. As with the first and second embodiments, bar 32 has a first end, a second end, and a central portion. A staple (not shown with this embodiment) is held in place in slot 36 (see especially FIG. 7C) by slideable sleeve 34 positioned over the second end of bar 32. Also shown in FIG. 7A are drill guides 38a & 38b that are positioned in drill guide holes 40a & 40b at the first end of bar 32. Angled rectangular posts 42 are provided on the first end of bar 32 to further facilitate the placement and/or removal of staples. FIGS. 7B-7D show various additional details of this third embodiment of the present invention in top plan, side, and bottom plan views of the surgical device.

FIG. 8A is a perspective view of a fourth embodiment of the surgical device of the present invention shown in a small sized version of the embodiment. The device of FIG. 8A may, for example, appropriately represent the geometry associated with a 9 mm (wide) staple version of the surgical device of the present invention. Tool 60 shown in FIG. 8A again generally comprises bar 62 with slideable sleeve 64. As before, bar 62 has a first end, a second end, and a central portion. Staple 66 is held in place in a slot (see FIG. 8C) by slideable sleeve 64 positioned over the second end of the bar 62. In the embodiment shown in FIG. 8A metal drill guides 68a & 68b are shown in position within drill guide holes 70a & 70b at the first end of the bar 62.

FIG. 8B shows a top plan view of the device shown in FIG. 8A showing the width of each of the components and the forward tilted orientation of staple 66 held within the second end of the bar 62. Drill guides 68a & 68b are shown extending from the opposite end of bar 62. FIG. 8D shows in greater detail the tab 67 that extends from sleeve 64 so as to facilitate the retention of staple 66 within the slot formed in bar 62. FIG. 8E is an end view of the first end of the bar 62 while FIG. 8F is an end view of the second end of the bar 62.

FIG. 8F shows the manner in which tab 67 extends from sleeve 64 so as to retain staple 66 within the slot (again, see the side view in FIG. 8C). FIG. 8G provides a cross-sectional view showing the manner in which sleeve 64 surrounds and slides over the main body of bar 62.

FIGS. 9A-9F are similar in most respects with the embodiment shown in FIGS. 8A-8G. The embodiment of FIGS. 9A-9F represent a larger sized tool on the order of 20 mm (for example). Otherwise, the structures and function of the tool remain basically the same as shown for the small version (9 mm) disclosed in FIGS. 8A-8G. FIG. 9A is a perspective view of a fifth embodiment of the surgical device of the present invention shown in a large sized version of the embodiment. The device of FIG. 9A may, for example, appropriately represent the geometry associated with a 20 mm (wide) staple version of the surgical device of the present invention. Tool 80 shown in FIG. 9A again generally comprises bar 82 with slideable sleeve 84. As before, bar 82 has a first end, a second end, and a central portion. Staple 86 is held in place in a slot (see FIG. 9C) by slideable sleeve 84 positioned over the second end of the bar 82. In the embodiment shown in FIG. 9A metal drill guides 88a & 88b are shown in position within drill guide holes 90a & 90b at the first end of the bar 82.

FIG. 9B shows a top plan view of the device shown in FIG. 9A showing the wider width of each of the components and again, the forward tilted orientation of staple 86 held within the second end of the bar 82. Drill guides 88a & 88b are shown extending from the opposite end of bar 82. FIG. 9D shows in greater detail the tab 87 that extends from sleeve 44 so as to facilitate the retention of staple 86 within the slot formed in bar 82. FIG. 9E is an end view of the first end of the bar 62. FIG. 8F provides a cross-sectional view showing the manner in which sleeve 84 surrounds and slides over the main body of bar 82.

In the various preferred embodiments of the present invention, the device may be manufactured in at least three sizes comprising small, medium, and large structures. Ultimately, the tool might be preferably be manufactured in six sizes including 9 mm, 11 mm, 13 mm, 15 mm, 18 mm, and 20 mm (staple widths). In each case, the size represents the width of the staple which is in turn reflected by the spacing on the drill guides and the width (length) of the slot into which the staple is positioned and held.

Reference is next made to FIGS. 10A-10C and FIGS. 11A-11C for a detailed description of an alternate embodiment of the sleeve configuration for the device of the present invention, as well as additional detail on the manner in which the staple is retained within the device and then released upon use. FIGS. 10A-10C show an alternate sleeve design configured on a tool with a staple in place. FIGS. 11A-11C represent the same tool but without the staple in place. In order to facilitate cost efficiency in the manufacture of the device, it may be preferable to mold a single sleeve capable of serving in conjunction with two different sized tool devices. The examples shown in FIGS. 10A-10C (and FIGS. 11A-11C) are structured to accommodate both 9 mm and 11 mm tools. Similar double tab sleeves might accommodate 13 mm and 15 mm tools or 18 mm and 20 mm tools. As shown in FIG. 10A, sleeve 92 incorporates two tabs, a first tab 93 (11 mm in this view) and a second smaller tab 94 (9 mm in this view). Sleeve 92 may be positioned on the tool in either orientation that allows the tab to function in association with retention of staple 96. In FIG. 10A, staple 96 (shown with its slightly S-shaped curve configuration) is retained in the slot formed in the bar of the tool. FIG. 11A shows the same structure with the staple removed disclosing the size and geometry of groove 98 into which the staple is secured and retained. Sliding sleeve 92 over the staple once it is positioned within groove 98 retains the staple in place until the user draws back the sleeve to release the staple.

Additionally shown in FIGS. 10A-10C (and FIGS. 11A-11C) is an alternate structure for the top surface of sleeve 92. The previous embodiments have shown a raised knurled surface that may be pushed or pulled by the user to release the staple. The embodiment shown in FIGS. 10A-10C (and FIGS. 11A-11C) provides a slight thumb depression suitable for carrying out the same sliding function. Such a sleeve configuration provides a lower profile and more readily accommodates the alternate use of two different tab widths as shown.

Various additional manufacturing efficiencies may be gained by providing various combinations of drill guide and staple retention structures on the ends of the bar of the tool which is held by the user for both the drilling and the staple placement functions.

Disclosed are materials, compositions, and components that can be used for, can be used in conjunction with, can be used in preparation for, or are products of the disclosed methods and devices. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a device is disclosed and discussed and a number of modifications that can be made to the device are discussed, each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed devices. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

A number of embodiments have been described. Nevertheless, it will be understood that various modifications may be made. Accordingly, other embodiments are within the scope of the claims.

I claim:

1. A surgical device comprising:
(a) a bar with a first and second end connected by a central arm, the first end of the bar comprising two drill guides;
(b) a slideable sleeve at the second end of the bar; and
(c) a staple comprising first and second legs and a middle portion between the two legs;
wherein the distance between the two drill guides is the same as the distance between the two legs of the staple, wherein the legs of the staple are perpendicular to the bar, and wherein the slidable sleeve is positionable in a first position at the second end of the bar over the middle portion of the staple and slidable to a second position toward the first end and over the central arm.

2. A method of implanting a staple into bone or bone fragment comprising:
(a) providing device comprising a bar with a first and second end connected by a central arm, the first end of the bar comprising two drill guides; a slideable sleeve at the second end of the bar, and a staple;
(b) placing the first end of the bar comprising the drill guides in a desired position over the bone or bone fragments where a staple is to be inserted;
(c) drilling through the drill guides two holes into the bone or bone fragments;
(d) placing the legs of the staples into the two drilled holes; and
(e) releasing the staple by sliding the slidable sleeve to the second position.

3. A surgical device comprising
(a) a bar with a first and second end connected by a central arm, wherein the first end of the bar comprises two drill guides having a width between them, and wherein the second end of the bar comprises a groove configured to hold a central portion of a staple, wherein the length of the groove corresponds to the distance between the drill guides; and
(b) a slideable sleeve at the second end of the bar, wherein the slideable sleeve comprises two notches on either side of a central portion, wherein each notch is configured to accommodate a staple leg, wherein the central portion between the two notches is configured to cover the groove in the second end of the bar;
wherein the slidable sleeve is positionable in a first position at the second end of the bar over the groove and slidable to a second position toward the first end and over the central arm.
